(19) United States
(12) Patent Application Publication
(10) Pub. No.: US 2018/0164320 A1
(43) Pub. Date: Jun. 14, 2018

(54) METHOD FOR DIAGNOSIS OF COLORECTAL CANCER USING MASS SPECTROMETRY OF N-GLYCANS

(71) Applicant: KOREA ADVANCED INSTITUTE OF SCIENCE AND TECHNOLOGY, Daejeon (KR)

(72) Inventors: Jung Hoe Kim, Daejeon (KR); Sung Hyeon Lee, Seoul (KR); Seung Yeol Park, Gyeonggi-do (KR); Hyun Joo An, Daejeon (KR); Jae-Han Kim, Daejeon (KR); Myung Jin Oh, Daejeon (KR); Jin Man Kim, Daejeon (KR); Ju A Lee, Seoul (KR)

(21) Appl. No.: 15/580,662

(22) PCT Filed: Dec. 16, 2015
(86) PCT No.: PCT/KR2015/013780

§ 371 Date: Dec. 7, 2017

(30) Foreign Application Priority Data
Jun. 11, 2015 (KR) 10-2015-0082751

(51) Int. Cl. G01N 33/574; G01N 30/86; G01N 30/72; C07H 5/04

(52) U.S. Cl. CPC G01N 33/57419; G01N 30/8631; G01N 2030/027; C07H 5/04; G01N 2560/00; G01N 30/7233

(57) ABSTRACT

The present invention relates to a method of diagnosing colorectal cancer by detection of glycan changes, and more particularly to a method of diagnosing colorectal cancer using mass spectrometry, in which, when specific glycan structures increase, decrease or significantly change due to a change in N-linked glycosylation of a colorectal cancer patient-derived glycoprotein, as detected by mass spectrometry, the glycan structures are selected as diagnostic markers.

### TECHNICAL FIELD

The present invention relates to a novel method of diagnosing colorectal cancer by detection of glycan changes and a method of detecting glycan changes to provide information for diagnosis of colorectal cancer, and more particularly to a method of diagnosing colorectal cancer using mass spectrometry of haptoglobin N-glycans detected by mass spectrometry, and to a method for detecting glycan changes.

### BACKGROUND ART

Cancer is the first leading cause of death in the world, including South Korea. Cancer is caused by genetic and environmental factors. Due to changed eating habits, severe environmental pollution, severe exposure to environmental and mental stress, etc., cancer development and the number of deaths by cancer are increasing each year. In comparison with other diseases, cancer is characterized in that complete cure is relatively difficult to achieve and in that post-treatment survival rate is relatively low. The characteristic of cancer in relation to survival rate is that the prognosis and survival rate of patients greatly differ depending on the degree of progression of cancer. Despite the advancement of cancer treatment technologies over about 100 years, the cure rate of terminal cancer or metastasized cancer is very low, even though it slightly differs depending on the kind of cancer (Etzioni R. et al., Nature Reviews Cancer 3, 243-252, 2003). Furthermore, cancer generally shows little or no subjective symptoms in an early stage, and if cancer is diagnosed by subjective symptoms, it is a cancer that had already progressed to a terminal stage, which is impossible to cure, in many cases. Namely, there is a need to develop cancer treatment methods, and a method capable of diagnosing cancer in an early stage in which the cancer can be cured can be considered a strategy that is most suitable for the purpose of effectively treating cancer and increasing survival rate. For this purpose, the research and development of biological factors (i.e., biomarkers) helpful in early diagnosis of cancer is currently being actively conducted based on proteomics techniques.

Tumor biomarkers are used in various applications. Namely, tumor can help in early diagnosis of cancer, and make it possible to determine the stage of cancer, monitor the progression of cancer during treatment, and determine prognosis after surgery (Rifai N. et al., Nature Biotech. 24, 971-983, 2006). In order to detect cancer and track the progression of cancer by biomarkers that are used for such purposes and that have such functions, nondestructive methods are required, and thus body fluids such as blood, which are tested at low risk, are recognized as optimal biological samples in the research and development of biomarkers. Namely, the development of biomarkers that can be detected in urine, saliva, blood or the like, is the most standardized approach, and among these body fluids, blood can be considered the most comprehensive biological sample in which proteins from all tissues are collected. Moreover, the form of tumor biomarker that is most preferable in terms of the form of biomaterial can be considered a protein.

Colorectal cancer refers to a malignant tumor occurring in the colon and the rectum. In the world, the incidence rate of colorectal cancer in 2000 (945,000 new cases; 9.6% of total cancers in the world) and the mortality of colorectal cancer in 2000 (492,000 deaths; 7.9% of total cancers) rank third among all cancers, and colorectal cancer develops in men and women at similar rates (men:women=1.1:1). Because colorectal cancer has relatively good prognosis compared to other cancers, the prevalence rate of colorectal cancer ranks second next to breast cancer in the world, and the number of persons who survive after diagnosed with colorectal cancer within the past five years is estimated to be about 2,400,000 (Parkin D M, Global cancer statistics in the year 2000, Lancet Oncol 2:533-543, 2001). In the prognosis of colorectal cancer, the five-year survival rate of early stage (stage 1) patients is 90% or more, whereas the five-year survival rate of metastasized (stage 4) colorectal cancer patients is only 5% (Cancer Facts and FIGS. 2004. American Cancer Society, 2004).

In South Korea, the incidence rate and mortality of colorectal cancer have recently significantly increased due to the Westernization of eating habits. According to Annual Report of the Korea Central Cancer Registry (January 2002 to December 2002) published by the Korean Ministry of Health and Welfare and the Korea Central Cancer Registry, 11,097 colorectal cancer cases occurred in 2002, and accounted for 11.2% of total cancer cases, which was the fourth highest among total cancers. The number of colorectal cancer cases in men was 6,423, which was larger than that in women (4,674). Moreover, the number of colorectal cancer cases in the 60s age group was the largest (3,751), and the number of colorectal cancer cases in the 50s age group was the second largest (2,400). According to four-year (from 1999 to 2002) data, the incidence rate of colorectal cancer increased steadily, and the crude incidence rate of colorectal cancer (the number of new cancer patients per 100,000 persons) increased from 22.5 in 1999 to 30.7 in 2002 (36.4% increase) in men, and increased from 18.8 in 1999 to 23.1 in 2002 (22.9% increase), and increased from 20.6 to 26.9 (30.6% increase) in men and women (cancer patient survival rate in 1993-2002 and cancer incidence rate in 1999-2002, the Korean Ministry of Health and Welfare, July 2007). In 2006, the number of deaths by colorectal cancer was a total of 6,277, which ranked fourth (9.5%) among total cancer deaths, and the number of deaths by colorectal cancer was 3,453 in men, which ranked fourth (8.0%), and was 2,824 in women, which ranked third (11.5%). In addition, colorectal cancer showed the highest increase in 10-year cancer mortality, next to lung cancer (2006 death and cause-of-death statistics, the National Statistical Office (NSO), Korea, September 2007).

In the case of colorectal cancer, progression from precancerous lesion or curable early-stage cancer is slow, and thus colorectal cancer screening makes it possible to reduce the incidence rate and mortality of the disease. It is believed that colorectal cancer screening for over 50-year-old men and women can reduce the rate of deaths caused by colorectal cancer (Walsh J M & Terdiman J P, JAMA 289:1288-96, 2003). However, at present, compliance and distribution rate for colonoscopy which is the most reliable screening method are low. On the contrary, a fecal occult blood test (FOBT), a noninvasive screening option which is currently most widely used, has several important limitations, including, inter alia, a problem of low sensitivity. In the USA, in 2002, only 40% of over 50-year old adults received colonoscopy within the past five years, and only 22% of over 50-year old adults received a fecal occult blood test (Behavior risk factor survey, National center for chronic disease prevention and health promotion. Centers for disease control and prevention, 2002). The reason why the rate of participation in colorectal cancer screening tests is lower than those in, particularly, breast cancer and cervical cancer screening tests, is because of various factors, including patient's discomfort, costs, insufficient recognition, low acceptance for current screening methods, etc.

Blood markers make it possible to efficiently diagnose colorectal cancer compared to feces markers, because analytical samples are easily obtained, patients can easily participate, samples are easily treated, and microorganisms capable of degrading biomarkers or interfering with analysis are not present. However, studies on biomarkers for diagnosis of colorectal cancer are still insufficient, and thus there is a need for the development of biomarkers for diagnosis of colorectal cancer.

Until now, conventional studies on the biochemical progression of cancer have been conducted with a focus on changes in protein expression. However, disease studies based on complex carbohydrate glycans that are biological components have not been properly conducted due to difficulty in the analysis of glycan structures. However, with the development of glycan structure analysis technology, glycan function analysis technology and glycan synthesis technology, the importance of complex carbohydrate glycans has been found rapidly, and there have been reports that the development of cancer cells is attributable to the role of various glycosyltransferases, and the resulting changes in the glycans of glycoprotein are associated with the carcinogenesis of cells (Orntoft, T. F., Vestergaard, E. M., “Clinical aspects of altered glycosylation of glycoproteins in cancer” Electrophoresis 1999, 20, 362-371).

Glycosylation is one of well-known post-translational modification processes. Cancer occurring in any organ has characteristic glycans, and thus glycans that are expressed as glycosphingolipids and glycoproteins are considered useful for favoring or inhibiting tumor development. Such glycans can be used for the purpose of diagnosis using various kinds of antibodies, and such glycan antigens are considered excellent targets for immunotherapy in various preclinical studies.

In South Korea, with changes in environmental factors, including the Westernization of eating habits, colorectal cancer shows a tendency to increase rapidly, and the age of persons diagnosed with colorectal cancer is also gradually decreasing. Thus, there is an increasing need for examination for early diagnosis, such as colonoscopy. Colorectal cancer shows its symptoms in a late stage, unlike the upper digestive organs, and even if the symptoms appear, colorectal cancer is misrecognized as constipation or piles, and thus proper treatment timing is missed in many cases. In South Korea, colorectal cancer ranks fourth next to gastric cancer, liver cancer and lung cancer, and shows a tendency to increase continuously.

In general cases, colorectal cancer patients show symptoms, including changed bowel habits, bloody stool, mucous stool (mucus feces), small-diameter feces, reduced body weight, abdominal discomfort (abdominal pain or abdominal distension), fatigue, inappetence, vomiting, nausea, anemia, etc. However, colorectal cancer does not show any symptoms in an early stage in most cases, and if any symptoms appear due to colorectal cancer, the colorectal cancer is one that had already significantly processed in many cases. Screening tests that are currently used for diagnosis of colorectal cancer include fecal occult blood tests, tumor marker tests, colonography, colonoscopy tests, computed tomography, abdominal ultrasonography, transrectal ultrasonography, and sigmoidoscopy, but such test methods have several limits to early diagnosis. In order to increase the survival rate of colorectal cancer patients, it is required to develop a method capable of early diagnosing colorectal cancer in a more accurate manner.

Due to the above-described problems, it is required to develop tumor biomarkers applicable to in vitro diagnostic techniques that can diagnose cancer using a small amount of body fluids, particularly blood. Until now, a colorectal cancer-related blood biomarker approved by the FDA has not yet been reported.

50% or more of human proteins are glycoproteins, and thus many human diseases are highly likely to be related to glycoproteins. Thus, it is possible to develop diagnostic markers by identifying disease-related glycoproteins and analyzing disease-specific glycan structures of the glycoproteins.

Although biochemical studies on most cancers have been focused on changes in the expression of proteins, the importance of complex carbohydrate glycans in cancer studies has increased with the development of glycan structure analysis technology. It is known that glycosylation that is one of post-translational modification processes can favor tumor development, but an accurate scientific basis for why glycan structures in tumors change has not yet been found. However, such cancer-specific glycans can be released into blood, and such glycans can be used for the purpose of diagnosis using various kinds of antibodies or the like. Plant-derived lectins can recognize various glycan structures, and such lectins are easily available and are low-priced, and thus are frequently used for the purpose of detecting glycan structures. However, lectins have a disadvantage in that they can detect only limited glycan structures. To overcome such problems, in recent years, developments have been made of methods capable of analyzing a very small amount of glycans, which could not be analyzed by conventional analytical methods, by use of an advanced mass spectrometer.

### DISCLOSURE OF INVENTION

### Technical Problem

An object of the present invention is to provide an excellent biomarker for diagnosis of colorectal cancer.

In addition, another object of the present invention is to provide a method for analyzing a colorectal cancer biomarker for the rapid and sensitive diagnosis of colorectal cancer.

### Technical Solution

In the present invention, blood glycoproteins containing glycans known to change from various kinds of cancers were isolated, and then colorectal cancer-specific glycans that are distinguished from those of normal persons were identified by mass spectrometry, and qualitative information and quantitative information about N-glycans obtained by treating purified glycoproteins with PNGase F were identified by mass spectrometry.

Abnormal glycosylation of haptoglobins from colorectal cancer patients was demonstrated by chip-based nano-LC/TOF-MS (Chip/TOF) spectrometry, after serum-derived haptoglobins were purified using antibody.

The results obtained in the present invention clearly indicate that some of N-glycan structures in serum haptoglobin derived from colorectal cancer patients are significantly less or more than those in a normal control (FIGS. 1A and 1B).

### Advantageous Effects

According to the present invention, several N-glycan structures having high sensitivity and high specificity, which are definitely different in the glycoproteins of a colorectal cancer patient group compared to those in a normal control group, can be simultaneously analyzed by mass spectrometry of the N-glycans of the glycoproteins of the colorectal cancer group. Furthermore, the present invention provides a method capable of diagnosing colorectal cancer by use of glycan structures, unlike a conventional method that analyzes only the amount of a certain protein.

### DESCRIPTION OF THE DRAWINGS

FIG. 1(a) shows the profile of total glycans analyzed by mass spectrometry of haptoglobins purified from the sera of normal persons and colorectal cancer patients, and is a graph showing the results of identifying glycan marker candidates showing a significant difference. Purified haptoglobins were treated with PNGase F to isolate only N-glycans, and then the N-glycans of haptoglobins derived from a normal control group and a colorectal cancer patient group were profiled by LC-MS. Each of glycan structures is shown as relative abundance, and all structures within the upper 95% of the total structures are shown. FIG. 1(b) shows three glycan structures showing the most significant difference among total glycans. High-mannose structures (5200, 6200 and 7200) were all identified to be potent colorectal cancer biomarker candidates showing an AUC 0.9 or more and a sensitivity and specificity of 80% or more.

### BEST MODE FOR CARRYING OUT THE INVENTION

Hereinafter, the configuration of the present invention will be described in more detail by way of Examples. However, it will be obvious to those skilled in the art that the scope of the present invention is not limited to only these Examples. In examples of the present invention, haptoglobin was used as an example of glycoprotein. However, it will be obvious to those skilled in the art that glycosylation-related enzymes are not glycosylated for certain proteins and that haptoglobin is described as an example of typical glycoprotein.

Materials and Other Reagents Anti-human beta-haptoglobin antibody was purchased from Dako (Carpinteria, Calif.). PNGase F (Peptide N-glycosidase F) was purchased from New England Biolabs (MA, USA). Graphitized carbon cartridges were purchased from Grace Davison Discovery Sciences (IL, USA). Mass spectrometry calculation (ESI-TOF Calibrant calibrant Mix mix G1969-85000) was performed using a product obtained from Agilent Technologies (CA, USA). All the chemicals used were of analytical grade or better.

Serum Samples from Colorectal Cancer Patients and Normal Persons

Serum samples were obtained from Chungnam National University Hospital (Korea), a member of the National Biobank of Korea. Clinical information about 20 colorectal cancer patients and 20 normal persons is summarized in Tables 1 and 2. The patients were biopsied and diagnosed by pathologists. This study was approved by the KAIST Institutional Review Board and was conducted under the consent of the participated normal persons and colorectal cancer patients.

Purification of Haptoglobin from Human Serum

Using anti-haptoglobin antibody, an anti-haptoglobin affinity column was prepared, and purification was performed. 500 μl of serum was obtained from each of 20 colorectal cancer patients and 20 normal persons and diluted in 4 ml, of PBS (phosphate-buffered saline, 10 mM phosphate buffer/2.7 mM KCl/137 mM NaCl, pH 7.4), and each of the dilutions was applied to the anti-haptoglobin affinity column and incubated in a rotating agitator at room temperature for 2 hours. Unbound materials were removed by washing the column with 30 ml, of PBS, and haptoglobin was eluted with elution buffer (0.1 M glycine/0.5 M NaCl, pH 2.8), and then fractionated into tubes containing neutralization buffer. The eluent was concentrated, and then centrifuged using a centrifugal filter (molecular weight cut-off: 10,000, Amicon Ultra, Millipore) to remove the surfactant, and then assayed for haptoglobin by a Quant-iT Assay Kit, after which it was subjected to 12.5% SDS-PAGE and Quant-iT Assay Kit (Invitrogen, Carlsbad, Calif.) and Coomassie blue staining. The samples were freeze-dried, and stored at −80° C. until use in analysis.

N-Glycan Isolation Using Enzyme

PNGase F (peptide N-glycosidase F; 500,000 unit/ml) derived from Flavobacterium meningosepticum was purchased from New England BioLabs (Ipswich, Mass.). To isolate glycans from protein by use of enzyme, 50 μl of the haptoglobin obtained in the above Example was dissolved in digestion buffer (pH 7.5, 100 mM ammonium bicarbonate, 5 mM DTT), and heated in boiling water for 2 minutes to denature the protein. After cooling at room temperature, 2 μl of PNGase F (1,000 units) was added thereto, and the mixture was incubated in a water bath at 37° C. for 16 hours.

400 μl of cold ethanol was added to the incubated mixture to precipitate the peptide and the protein.

The resulting solution was frozen at −40° C. for 60 minutes, and then centrifuged at 14,000 rpm and 4° C. for 20 minutes. Next, for each sample, 400 μl of the supernatant was collected, and ethanol contained in the supernatant was completely dried.

Thereafter, 1 ml of water was added to each sample, followed by intensive stirring, thereby preparing glycan-containing samples for purification.

Glycan Purification

Each of the glycan-containing samples isolated by PNGase F was purified by a graphitized carbon cartridge SPE (PGC-SPE; packing amount: 150 mg; cartridge volume: 3 ml). The PGC SPE cartridge was obtained from Alltech (Deerfield, Ill.). Prior to use, the cartridge was washed with 6 ml of ultrapure water, and washed with 6 ml of 80% (v/v) acetonitrile (ACN) containing 0.1% trifluoroacetic acid (TFA), followed by washing with 6 ml of ultrapure water. The glycan-containing sample was placed in the PGC cartridge, and a several-fold volume of ultrapure water was allowed to flow through the cartridge at a rate of 1 ml/min to remove salts. N-glycans were eluted sequentially with 10% (v/v) acetonitrile, 20% (v/v) acetonitrile, and 40% (v/v) acetonitrile plus 0.05% (v/v) TFA. Each of the fractions was collected and dried with a centrifugal evaporator. The fractions were dissolved in ultrapure water before mass spectrometry.

Chip-Based Nano-LC/MS and MS/MS

Nano-LC separation was performed according to conventional technology. The N-glycan fractions for each sample were combined with each other, and 2.0 μl (corresponding to 800 ng of haptoglobin) was loaded onto a nano-LC column (Agilent Technologies) having a chip placed thereon by an autosampler. The nano-LC column consists of an enrichment column (9×0.075 mm I.D.) and an analytical column (43×0.075 mm I.D.), both packed with 5 μm porous graphitized carbon as the stationary phase. A rapid glycan elution gradient was delivered at a rate of 0.3 μl/min using solutions of (A) 3.0% acetonitrile and 0.1% formic acid (v/v) in water, and (B) 90.0% acetonitrile and 0.1% formic acid (v/v) in water, ramping from 6% to 100% B solution over 20 minutes. Remaining non-glycan compounds were flushed out with 100% B solution prior to re-equilibration. After chromatographic separation, glycans were ionized by a chip-integrated nano-ESI spray tip and analyzed by a Q-TOF mass analyzer (Model 6540, Agilent Technologies) according to conventional technology. Calibrant molecules (ESI-TOF Calibrant Mix G1969-85000, Agilent Technologies) were injected directly into an electrospray mass spectrometer to make internal mass measurement possible. MS spectra were acquired in positive ionization mode over a mass range of m/z 500-2000 with an acquisition time of 2 seconds per spectrum. MS/MS spectra were acquired in positive ionization mode over a mass range of m/z 100-3000 with an acquisition time of 1.5 seconds per spectrum. Following an MS scan, precursor compounds were automatically selected for MS/MS analysis by the acquisition software based on ion abundance and charge state (z=2 or 3) and isolated in the quadrupole with a mass bandpass FWHM (full width at half maximum) of 1.3 m/z. Collision energies for CID fragmentation were calculated for each precursor compound based on the following formula:

V_{collision}=1.8V{(m/z)/100 Da}−4.8V

wherein V_{collision} is the potential difference applied across the collision cell to accelerate and fragment the precursor. Raw LC/MS date was processed using the Molecular Feature Extractor algorithm included in the MassHunter Qualitative Analysis software (version B.04.00 SP2, Agilent Technologies). MS peaks were filtered with a signal-to-noise ratio of 5.0 and deconvoluted to create a list of compound mass, ion abundance and retention time.

Identification of N-Glycans by Accurate Mass

The compounds identified by nano-LC/MS were matched by accurate mass to a glycan database that covers all possible complex, hybrid, and high-mannose glycan compositions based on known biological synthesis pathways and glycosylation patterns. Deconvoluted mass of each ECC peak were compared against theoretical glycan mass using a mass error tolerance of 20 ppm. As the sample set originated from human serum, only glycan compositions containing hexose, HexNAc (N-acetylhexosamine), fucose and NeuAc (N-acetylneuraminic acid) were considered. Using T-test p-value analysis, receiver-operating characteristic (ROC) curve and AUC (Area under the ROC curve), N-glycans extracted from each sample were comparatively analyzed.

Results 1: Analysis of Colorectal Cancer-Specific N-Glycans of Haptoglobin

Detailed glycosylation patterns of the blood glycoprotein haptoglobin were analyzed by a chip-based nano-LC/TOF-MS (Chip/TOF) system. This system can identify the heterogeneity of glycans having different connection or antennary structures, and can provide higher sensitivity than MALDI-MS and conventional LC/MS, because of additional advantages such as the provision of low energy ion, large dynamic range and unmatched retention time reproducibility. In the present invention, the N-glycans of haptoglobins derived from the sera of normal persons and patients (n=40) were analyzed twice by nano-LC/MS. Only the N-glycans of haptoglobins were separated by PNGase F treatment, and then the N-glycans of haptoglobins derived from the normal control group and the colorectal cancer patient group were compared with one another by chip-based nano-/TOF-MS (Chip/TOF). All structures within the upper 95% of total N-glycan structures found in each sample were used, and quantitative values were compared with one another. Among high-mannose structures of the N-glycan structures, Hex5-HexNAc2 (5200 glycan structure) showing a mass value of 1234.43, Hex6-HexNAc2 (6200 glycan structure) showing a mass value of 1396.48, and Hex7-HexNAc2 (7200 glycan structure) showing a mass value of 1558.54, etc., showed an AUC value of 0.90 or higher. Furthermore, among several glycan structures showing a significant difference between the normal control group and the colorectal cancer patient group, Hex4-HexNAc3-NeuAc1 glycan (1566.6 m/z), Hex5-HexNAc5-Fuc1-NeuAc2 glycan (2571.9 m/z), Hex6-HexNAc5 glycan (2005.7 m/z), and Hex6-HexNAc5-NeuAc2 glycan (2587.9 m/z) structures in addition to high-mannose structures showed a difference between the colorectal cancer sample and the normal control group (P<0.01), but showed no difference between a gastric cancer patient group and the normal control group.

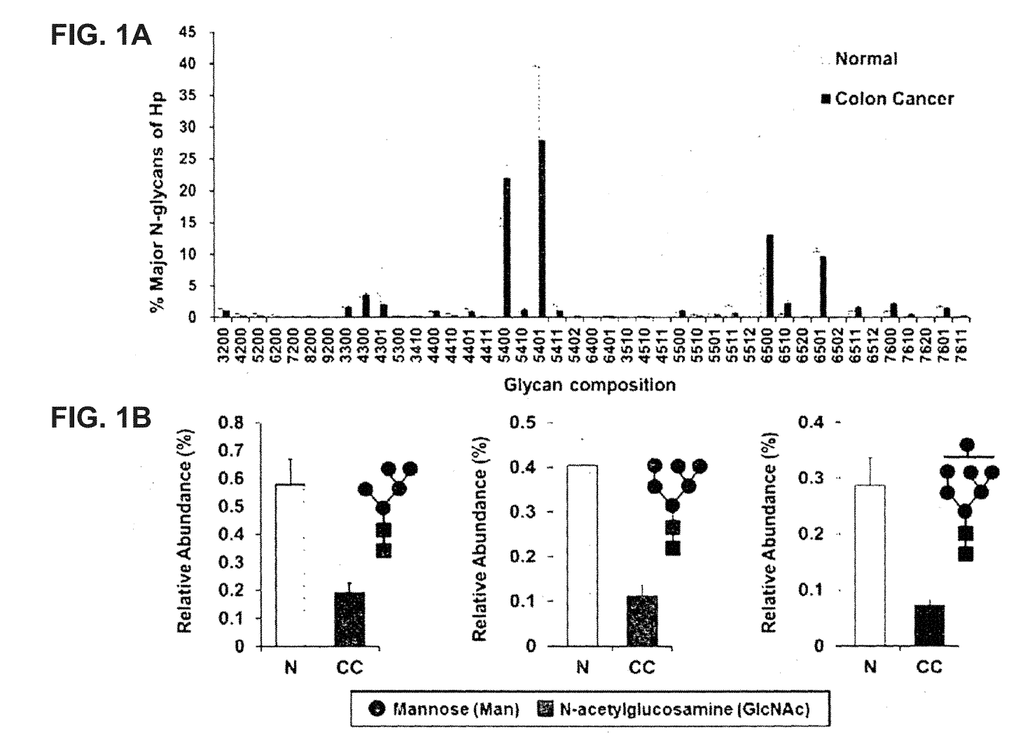

FIG. 1 shows the profile of total glycans analyzed by mass spectrometry of haptoglobins purified from the sera of normal persons and colorectal cancer patients, and is a graph showing the results of identifying glycan marker candidates showing a significant difference from the date. FIG. 1A shows the results obtained by treating purified haptoglobins with PNgase F to separate only N-glycans, and then profiling the N-glycans of haptoglobins, derived from a normal control group and a colorectal cancer patient group, by LC-MS. Each of N-glycan structures is shown as relative abundance, and all structures within the upper 95% of the total structures are shown. FIG. 1B shows three N-glycan structures showing the most significant difference among total N-glycans. High-mannose structures (5200, 6200 and 7200) were all identified to be potent colorectal cancer biomarker candidates showing an AUC 0.9 or higher and a sensitivity and specificity of 80% or higher.

Tables 1 and 2 show summary information about a total of 40 normal persons and colorectal cancer patients (20 normal persons and 20 colorectal cancer patients). Serum samples were obtained from Chungnam National University Hospital (Korea), a member of the National Biobank of Korea. The patients were biopsied and diagnosed by pathologists.

Tables 3 and 4 show a list of N-glycan structures showing a sensitivity corresponding to a p value of 0.05 or less between the normal control group and the colorectal cancer patient group, among N-glycan structures separated from the haptoglobins identified by nano LC chip/Q-TOF MS spectrometry. For example, N-chain structures can be identified based on a retention time library, and the amounts of all haptoglobin-derived N-chain structures can be determined. In Tables 3 and 4, glycan structures are classified into high-mannose structures and antennary structures, based on the results of mass (MS) mass spectrometry. Particularly, it was shown that several high-mannose structures (Hex5-HexNAc2 glycan (1234.4 m/z), Hex6-HexNAc2 glycan (1396.5 m/z), Hex7-HexNAc2 glycan (1558.5 m/z)) showed an AUC of 0.9 or higher, and thus could accurately distinguish the colorectal cancer patients from the normal persons. In addition, a Hex4-HexNAc3-NeuAc1 glycan (1566.6 m/z), Hex5-HexNAc5-Fuc1-NeuAc2 glycan (2571.9 m/z), Hex6-HexNAc5 glycan (2005.7 m/z), and Hex6-HexNAc5-NeuAc2 glycan (2587.9 m/z) structures showed a difference between the colorectal cancer samples and the normal control group (P<0.01), but showed no difference between gastric cancer patient samples and the normal control groups, indicating that it is a colorectal cancer-specific biomarker candidate distinguishable from gastric cancer.

The present invention uses body fluids such as serum as samples, and particularly, uses haptoglobins that are glycoproteins present in serum in large amounts. Thus, the present invention can be easily applied to in vitro diagnostic technology. Colorectal cancer-related biomarkers currently approved by the FDA include CEA protein, but the CEA protein has a limitation in that it shows a detection rate of about 70%.

The present invention can provide a method of diagnosing colorectal cancer based on the difference in expression of specific N-glycan structures (including high-mannose structures) between colorectal cancer patients and normal persons by analyzing N-glycan structures, which change in colorectal cancer patients compared to those in normal persons, by mass spectrometry. Glycan biomarkers, including high-mannose structures, identified by mass spectrometry, are listed in Tables 3 and 4. These glycan structures are biomarkers having a significance of p=0.05 or less.

The present invention can be applied for the development of a new method for diagnosis of colorectal cancer, a composition for diagnosis of colorectal cancer, and a kit for diagnosis of colorectal cancer.

TABLE 1

| Case | | | | | | |
|---|---|---|---|---|---|---|
| no. | Classification | Age | Sex | Location | ClinicalTNM | Stage |
| cc1 | Colon Cancer | 71 | F | ascending | T2N0M0 | I |
| Cc2 | Colon Cancer | 51 | F | sigmoid | T3N0M0 | II A |
| Cc3 | Colon Cancer | 70 | F | rectum | T3N0M0 | II A |
| Cc4 | Colon Cancer | 54 | F | descending | T2N0M0 | I |
| Cc5 | Colon Cancer | 62 | F | sigmoid | T3N0M0 | II A |
| Cc6 | Colon Cancer | 81 | M | ascending | T3N0M0 | II A |
| Cc7 | Colon Cancer | 68 | F | ascending | T1N0M0 | I |
| Cc8 | Colon Cancer | 65 | M | rectum | T3N0M0 | II A |
| Cc9 | Colon Cancer | 73 | M | rectum | T3N1bM0 | III B |
| Cc10 | Colon Cancer | 64 | F | ascending | T3N0M0 | II A |
| cc11 | Colon Cancer | 73 | F | rectosigmoid | T3N0M0 | II A |
| cc12 | Colon Cancer | 76 | F | rectum | T0N0M0 | III B |
| cc13 | Colon Cancer | 69 | F | sigmoid | TisN0M0 | 0 |
| cc14 | Colon Cancer | 62 | M | sigmoid | T3N0M0 | II A |
| cc15 | Colon Cancer | 64 | M | ascending | T2N0M0 | I |
| cc16 | Colon Cancer | 61 | M | rectum | T2N0M0 | I |
| cc17 | Colon Cancer | 70 | F | ascending | T2N0M0 | I |
| cc18 | Colon Cancer | 66 | F | sigmoid | T3N1M1 | IV |
| cc19 | Colon Cancer | 68 | F | sigmoid | TN0M0 | I |
| cc20 | Colon Cancer | 70 | F | rectum | T2N0M0 | I |

TABLE 2

| Case no. | Classification | Age | Sex |
|---|---|---|---|
| n1 | normal | 34 | M |
| n2 | normal | 31 | M |
| n3 | normal | 31 | M |
| n4 | normal | 30 | M |
| n5 | normal | 53 | F |
| n6 | normal | 49 | F |
| n7 | normal | 49 | F |
| n8 | normal | 51 | F |
| n9 | normal | 49 | F |
| n10 | normal | 61 | F |
| n11 | normal | 61 | F |
| n12 | normal | 60 | M |
| n13 | normal | 34 | F |
| n14 | normal | 52 | F |
| n15 | normal | 51 | M |
| n16 | normal | 59 | M |
| n17 | normal | 47 | F |
| n18 | normal | 63 | F |
| n19 | normal | 46 | M |
| n20 | normal | 47 | M |

TABLE 3

| | | | | | Relative | | | |
|---|---|---|---|---|---|---|---|---|
| | | | | | abundance(%) | | | |
| | Composition | | | | | Colon | | |
| GlycanMass/Da | Hex | HexNAc | Fuc | NeuAc | Normal | Cancer | t-Test | AUC |
| High Mannose | | | | | | | | |
| 1234.43 | 5 | 2 | 0 | 0 | 0.58 | 0.19 | 0.000555 | 0.91 |
| 1396.48 | 6 | 2 | 0 | 0 | 0.40 | 0.11 | 0.000108 | 0.91 |
| 1558.54 | 7 | 2 | 0 | 0 | 0.29 | 0.07 | 0.000377 | 0.94 |
| 1720.59 | 8 | 2 | 0 | 0 | 0.27 | 0.08 | 0.008656 | 0.87 |
| 1882.65 | 9 | 2 | 0 | 0 | 0.10 | 0.03 | 0.000204 | 0.83 |
| Mono, Bi-antennary | | | | | | | | |
| 1566.56 | 4 | 3 | 0 | 1 | 3.4865 | 2.065 | 0.009169 | 0.77 |
| 1437.50 | 5 | 3 | 0 | 0 | 0.2468 | 0.1444 | 0.003803 | 0.75 |
| 1462.54 | 3 | 4 | 1 | 0 | 0.2645 | 0.1235 | 0.006786 | 0.74 |
| 1624.60 | 4 | 4 | 1 | 0 | 0.5336 | 0.2776 | 0.010488 | 0.72 |
| 1769.64 | 4 | 4 | 0 | 1 | 1.3232 | 0.8425 | 0.025722 | 0.76 |
| 1915.71 | 4 | 4 | 1 | 1 | 0.1402 | 0.0325 | 0.001757 | 0.83 |
| 1640.59 | 5 | 4 | 0 | 0 | 14.3 | 21.967 | 0.004744 | 0.77 |
| 1786.65 | 5 | 4 | 1 | 0 | 0.7582 | 1.2421 | 0.021130 | 0.65 |
| 1931.69 | 5 | 4 | 0 | 1 | 39.632 | 27.961 | 0.018485 | 0.77 |
| 2077.74 | 5 | 4 | 1 | 1 | 1.8768 | 0.9942 | 0.000309 | 0.85 |
| 1802.65 | 6 | 4 | 0 | 0 | 0.0261 | 0.0975 | 0.000467 | 0.72 |

TABLE 4

| | | | | | Relative | | | |
|---|---|---|---|---|---|---|---|---|
| | | | | | abundance(%) | | | |
| | Composition | | | | | Colon | | |
| GlycanMass/Da | Hex | HexNAc | Fuc | NeuAc | Normal | Cancer | t-Test | AUC |
| Tri-antennary | | | | | | | | |
| 1665.63 | 3 | 5 | 1 | 0 | 0.1043 | 0.0532 | 0.012200 | 0.69 |
| 1827.65 | 4 | 5 | 1 | 0 | 0.2608 | 0.134 | 0.007611 | 0.77 |
| 1989.73 | 5 | 5 | 1 | 0 | 0.4789 | 0.2619 | 0.000844 | 0.8 |
| 2134.76 | 5 | 5 | 0 | 1 | 0.7312 | 0.4441 | 0.000218 | 0.86 |
| 2280.82 | 5 | 5 | 1 | 1 | 1.8226 | 0.6526 | 0.000262 | 0.9 |
| 2571.92 | 5 | 5 | 1 | 2 | 0.3514 | 0.0355 | 0.000710 | 0.86 |
| 2005.72 | 6 | 5 | 0 | 0 | 6.7373 | 13.096 | 0.002537 | 0.8 |
| 2151.77 | 6 | 5 | 1 | 0 | 0.4991 | 2.2004 | 0.000539 | 0.79 |
| 2297.85 | 6 | 5 | 2 | 0 | 0.0269 | 0.1298 | 0.015538 | 0.6 |
| 2587.93 | 6 | 5 | 0 | 2 | 0.082 | 0.0206 | 0.003398 | 0.77 |
| 2442.88 | 6 | 5 | 1 | 1 | 1.0125 | 1.6435 | 0.012092 | 0.72 |
| Tetra-antennary | | | | | | | | |
| 2370.85 | 7 | 6 | 0 | 0 | 0.9597 | 2.1479 | 0.001962 | 0.75 |
| 2516.91 | 7 | 6 | 1 | 0 | 0.0566 | 0.4507 | 0.003370 | 0.74 |
| 2808.00 | 7 | 6 | 1 | 1 | 0.089 | 0.2639 | 0.005788 | 0.73 |

Haptoglobin is one of highly abundant glycoproteins, and is an acute phase protein that increases in the progression of various diseases such as inflammation and tumors. It is known that haptoglobin has four N-glycosylation sites at asparagines 184, 207, 211 and 241 and has one O-glycosylation site. A particular glycosylation type and a particular glycosylation site, which provides glycan changes that are distinguished between colorectal cancer patients and normal persons, are not known.

The present inventors performed the purification of serum-derived haptoglobin by anti-haptoglobin antibody affinity chromatography.

The present inventors determined an exact glycosylation state by chip-based nano-LC/TOF-MS (Chip/TOF) spectrometry following immune affinity chromatography purification. Because LC-MS causes increased sensitivity and less ion fragmentation compared to MALDI-MS, the present inventors could successfully demonstrate the detailed glycan structures of haptoglobins. In conclusion, modified N-glycans were detected in haptoglobins derived from colorectal cancer patients.

Several glycan structures showing a significant difference between a normal control group and a colorectal cancer patient group could be found by glycan structure profiling. Various N-glycan structures, including high-mannose structures, showed a significant difference in relative amount between the normal control group and the colorectal cancer patient group (Tables 3 and 4).

The present inventors have found the difference in high-mannose structures between the normal control group and the colorectal cancer patient group. Interestingly, this difference in high-mannose structures was observed by chip-based nano-LC/TOF-MS (Chip/TOF), because the use of this method could classify glycan structures with high sensitivity. This fact demonstrates that the high-sensitivity mass spectrometry method can be effectively used for diagnosis of cancer by use of biomarkers. Such results suggest that the abnormal glycan structures obtained in the present invention are useful glycan biomarkers that can replace current nonspecific colorectal cancer markers.

The present invention is related to a method for analyzing a colorectal cancer biomarker comprises:
(a) isolating a haptoglobin from a subject-derived blood sample;
(b) isolating a N-glycan from the isolated haptoglobin;
(c) analyzing mass of the isolated N-glycan by LC/MS analysis; and
(d) determining the structure and composition of the N-glycan, and performing quantitative profiling of the N-glycan based on the results of the LC/MS analysis.

In addition, the N-glycan in step (d) may be at least one selected from the group consisting of
Hex5-HexNAc2 glycan (1234.4 m/z),
Hex6-HexNAc2 glycan (1396.5 m/z),
Hex7-HexNAc2 glycan (1558.5 m/z),
Hex8-HexNAc2 glycan (1720.6 m/z),
Hex9-HexNAc2 glycan (1882.7 m/z),
Hex4-HexNAc3-NeuAc1 glycan (1566.6 m/z),
Hex5-HexNAc3 glycan (1437.5 m/z),
Hex3-HexNAc4-Fuc1 glycan (1462.5 m/z),
Hex4-HexNAc4-Fuc1 glycan (1624.6 m/z),
Hex4-HexNAc4-NeuAc1 glycan (1769.6 m/z),
Hex4-HexNAc4-Fuc1-NeuAc1 glycan (1915.7 m/z),
Hex5-HexNAc4 glycan (1640.6 m/z),
Hex5-HexNAc4-Fuc1 glycan (1786.7 m/z),
Hex5-HexNAc4-NeuAc1 glycan (1931.7 m/z),
Hex5-HexNAc4-Fuc1-NeuAc1 glycan (2077.7 m/z),
Hex6-HexNAc4 glycan (1802.7 m/z),
Hex3-HexNAc5-Fuc1 glycan (1665.6 m/z),
Hex4-HexNAc5-Fuc1 glycan (1827.7 m/z),
Hex5-HexNAc5-Fuc1 glycan (1989.7 m/z),
Hex5-HexNAc5-NeuAc1 glycan (2134.8 m/z),
Hex5-HexNAc5-Fuc1-NeuAc1 glycan (2280.8 m/z),
Hex5-HexNAc5-Fuc1-NeuAc2 glycan (2571.9 m/z),
Hex6-HexNAc5 glycan (2005.7 m/z),
Hex6-HexNAc5-Fuc1 glycan (2151.8 m/z),
Hex6-HexNAc5-Fuc2 glycan (2297.9 m/z),
Hex6-HexNAc5-NeuAc2 glycan (2587.9 m/z),
Hex6-HexNAc5-Fuc1-NeuAc1 glycan (2442.9 m/z),
Hex7-HexNAc6 glycan (2370.9 m/z),
Hex7-HexNAc6-Fuc1 glycan (2516.9 m/z), and
Hex7-HexNAc6-Fuc1-NeuAc1 glycan (2808.0 m/z).

In addition, the N-glycan in step (d) may be at least one selected from the group consisting of Hex4-HexNAc3-NeuAc1 glycan (1566.6 m/z), Hex5-HexNAc5-Fuc1-NeuAc2 glycan (2571.9 m/z), Hex6-HexNAc5 glycan (2005.7 m/z), and Hex6-HexNAc5-NeuAc2 glycan (2587.9 m/z).

The structures of the N-glycans show a difference between a colorectal cancer sample and a normal sample (P<0.01), but no difference between a gastric cancer sample and a normal sample.

In addition, the N-glycan in step (d) may be at least one selected from the group consisting of Hex5-HexNAc2 glycan (1234.4 m/z), Hex6-HexNAc2 glycan (1396.5 m/z), Hex7-HexNAc2 glycan (1558.5 m/z), Hex8-HexNAc2 glycan (1720.6 m/z), and Hex9-HexNAc2 glycan (1882.7 m/z).

In addition, the LC/MS analysis in step (c) may be nano-LC chip/Q-TOF mass spectrometry (MS).

In addition, the quantitative profiling in step (d) may be performed using at least one selected from the group consisting of T-test p-value analysis, ROC (Receiver-Operating Curve) analysis, and AUC (Area under the ROC curve) analysis

In addition, the blood sample may be whole blood, serum, or plasma.

The present invention is also related to a method for analyzing a colorectal cancer biomarker comprises:
(a) isolating a haptoglobin from each of a subject-derived blood sample and a normal blood sample;
(b) isolating N-glycans from each of the isolated haptoglobins;
(c) analyzing mass of the isolated N-glycans by LC/MS analysis;
(d) determining the structure and composition of the N-glycans, and performing quantitative profiling of the N-glycans based on the results of the LC/MS analysis; and
(e) selecting the N-glycan derived from the subject-derived blood sample as the colorectal cancer biomarker, when the N-glycan derived from the subject-derived blood sample has either a T-test p-value of 0.05 or less compared to that of the N-glycan derived from the normal blood sample, or an AUC (Area under the ROC curve) value of 0.7 or higher.

In addition, the method for analyzing a colorectal cancer biomarker further comprise, after step (e), step (f) of determining the subject has colorectal cancer when the content of the subject sample-derived N-glycan which is selected as the colorectal cancer biomarker has a significant difference from the content of the normal blood sample-derived N-glycan.

In addition, the selected colorectal cancer biomarker in step (e) may be at least one selected from the group consisting of
Hex5-HexNAc2 glycan (1234.4 m/z),
Hex6-HexNAc2 glycan (1396.5 m/z),
Hex7-HexNAc2 glycan (1558.5 m/z),
Hex8-HexNAc2 glycan (1720.6 m/z),
Hex9-HexNAc2 glycan (1882.7 m/z),
Hex4-HexNAc3-NeuAc1 glycan (1566.6 m/z),
Hex5-HexNAc3 glycan (1437.5 m/z),
Hex3-HexNAc4-Fuc1 glycan (1462.5 m/z),
Hex4-HexNAc4-Fuc1 glycan (1624.6 m/z),
Hex4-HexNAc4-NeuAc1 glycan (1769.6 m/z),
Hex4-HexNAc4-Fuc1-NeuAc1 glycan (1915.7 m/z),
Hex5-HexNAc4 glycan (1640.6 m/z),
Hex5-HexNAc4-Fuc1 glycan (1786.7 m/z),
Hex5-HexNAc4-NeuAc1 glycan (1931.7 m/z),
Hex5-HexNAc4-Fuc1-NeuAc1 glycan (2077.7 m/z),
Hex6-HexNAc4 glycan (1802.7 m/z),
Hex3-HexNAc5-Fuc1 glycan (1665.6 m/z),
Hex4-HexNAc5-Fuc1 glycan (1827.7 m/z),
Hex5-HexNAc5-Fuc1 glycan (1989.7 m/z),
Hex5-HexNAc5-NeuAc1 glycan (2134.8 m/z),
Hex5-HexNAc5-Fuc1-NeuAc1 glycan (2280.8 m/z),
Hex5-HexNAc5-Fuc1-NeuAc2 glycan (2571.9 m/z),
Hex6-HexNAc5 glycan (2005.7 m/z),
Hex6-HexNAc5-Fuc1 glycan (2151.8 m/z),
Hex6-HexNAc5-Fuc2 glycan (2297.9 m/z),
Hex6-HexNAc5-NeuAc2 glycan (2587.9 m/z),
Hex6-HexNAc5-Fuc1-NeuAc1 glycan (2442.9 m/z),
Hex7-HexNAc6 glycan (2370.9 m/z),
Hex7-HexNAc6-Fuc1 glycan (2516.9 m/z), and
Hex7-HexNAc6-Fuc1-NeuAc1 glycan (2808.0 m/z).

In addition, the selected colorectal cancer biomarker in step (e) may be at least one selected from the group consisting Hex5-HexNAc2 glycan (1234.4 m/z), Hex6-HexNAc2 glycan (1396.5 m/z), Hex7-HexNAc2 glycan (1558.5 m/z), Hex8-HexNAc2 glycan (1720.6 m/z), and Hex9-HexNAc2 glycan (1882.7 m/z).

In addition, the selected colorectal cancer biomarker in step (e) may be at least one selected from the group consisting Hex4-HexNAc3-NeuAc1 glycan (1566.6 m/z), Hex5-HexNAc5-Fuc1-NeuAc2 glycan (2571.9 m/z), Hex6-HexNAc5 glycan (2005.7 m/z), and Hex6-HexNAc5-NeuAc2 glycan (2587.9 m/z).

In addition, the blood sample may be whole blood, serum, or plasma.

### INDUSTRIAL APPLICABILITY

As described above, the present invention can be used for the diagnosis and prevention of colorectal cancer.

1.-13. (canceled)

14. A method for diagnosing a colorectal cancer, comprising:
(a) measuring a content of at least one biomarker selected from the group consisting of
Hex5-HexNAc2 glycan (1234.4 m/z),
Hex6-HexNAc2 glycan (1396.5 m/z),
Hex7-HexNAc2 glycan (1558.5 m/z),
Hex8-HexNAc2 glycan (1720.6 m/z),
Hex9-HexNAc2 glycan (1882.7 m/z),
Hex4-HexNAc3-NeuAc1 glycan (1566.6 m/z),
Hex5-HexNAc3 glycan (1437.5 m/z),
Hex3-HexNAc4-Fuc1 glycan (1462.5 m/z),
Hex4-HexNAc4-Fuc1 glycan (1624.6 m/z),
Hex4-HexNAc4-NeuAc1 glycan (1769.6 m/z),
Hex4-HexNAc4-Fuc1-NeuAc1 glycan (1915.7 m/z),
Hex5-HexNAc4 glycan (1640.6 m/z),
Hex5-HexNAc4-Fuc1 glycan (1786.7 m/z),
Hex5-HexNAc4-NeuAc1 glycan (1931.7 m/z),
Hex5-HexNAc4-Fuc1-NeuAc1 glycan (2077.7 m/z),
Hex6-HexNAc4 glycan (1802.7 m/z),
Hex3-HexNAc5-Fuc1 glycan (1665.6 m/z),
Hex4-HexNAc5-Fuc1 glycan (1827.7 m/z),
Hex5-HexNAc5-Fuc1 glycan (1989.7 m/z),
Hex5-HexNAc5-NeuAc1 glycan (2134.8 m/z),
Hex5-HexNAc5-Fuc1-NeuAc1 glycan (2280.8 m/z),
Hex5-HexNAc5-Fuc1-NeuAc2 glycan (2571.9 m/z),
Hex6-HexNAc5 glycan (2005.7 m/z),
Hex6-HexNAc5-Fuc1 glycan (2151.8 m/z),
Hex6-HexNAc5-Fuc2 glycan (2297.9 m/z),
Hex6-HexNAc5-NeuAc2 glycan (2587.9 m/z),
Hex6-HexNAc5-Fuc1-NeuAc1 glycan (2442.9 m/z),
Hex7-HexNAc6 glycan (2370.9 m/z),
Hex7-HexNAc6-Fuc1 glycan (2516.9 m/z), and
Hex7-HexNAc6-Fuc1-NeuAc1 glycan (2808.0 m/z), from a subject-derived blood sample;
(b) diagnosing the subject to have colorectal cancer, when the content of the biomarker derived from the subject-derived blood sample has a T-test p-value of 0.05 or less, or an AUC (Area under the ROC curve) value of 0.7 or higher, compared to that derived from a normal blood sample.

15. The method of claim 14, wherein the colorectal cancer biomarker is at least one selected from the group consisting
Hex5-HexNAc2 glycan (1234.4 m/z),
Hex6-HexNAc2 glycan (1396.5 m/z),
Hex7-HexNAc2 glycan (1558.5 m/z),
Hex8-HexNAc2 glycan (1720.6 m/z), and
Hex9-HexNAc2 glycan (1882.7 m/z).

16. The method of claim 14, wherein the colorectal cancer biomarker is at least one selected from the group consisting
Hex4-HexNAc3-NeuAc1 glycan (1566.6 m/z),
Hex5-HexNAc5-Fuc1-NeuAc2 glycan (2571.9 m/z),
Hex6-HexNAc5 glycan (2005.7 m/z), and
Hex6-HexNAc5-NeuAc2 glycan (2587.9 m/z).

17. The method of claim 14, wherein the content of a biomarker in step (a) is analyzed by LC/MS analysis.

18. The method of claim 14, wherein the LC/MS analysis is nano-LC chip/Q-TOF mass spectrometry (MS).

19. The method of claim 14, wherein the blood sample is whole blood, serum, or plasma.
